(19) United States
(12) Patent Application Publication
(10) Pub. No.: US 2005/0186255 A1
(43) Pub. Date: Aug. 25, 2005

(54) Feminine wipe for symptomatic treatment of vaginitis

(76) Inventor: Syed Rizvi, Bakersfield, CA (US)

Correspondence Address: DONALD A. KETTLESTRINGS, 414 HUNGERFORD DRIVE, SUITE 211, ROCKVILLE, MD, 20850, US

(21) Appl. No.: 10/780,661

(22) Filed: Feb. 19, 2004

(51) Int. Cl. A61K 9/70

(52) U.S. Cl. 424/443

(57) ABSTRACT

An article and method are described for treatment of vaginitis to inhibit bacterial growth and other odor causing and infectious organisms in the genital area. The composition is a liquid which conveniently is impregnated onto an absorbent substrate such as a fabric patch, which may be enclosed in a sealed envelope.

This application claims the benefit of U.S. Provisional Application No. 60/339,399, filed Dec. 14, 2001.

### FIELD OF INVENTION

The present invention relates to a composition and wipe for symptomatic relief of vaginitis. This composition specifically provides relief from discharge and odor which are the common symptoms of vaginitis. Without treatment of the underlying cause, women who suffer from these unpleasant symptoms, have restricted daily routine activities.

### BACKGROUND OF THE INVENTION

Vaginal odor is caused by the production of several chemicals, particularly amines, by bacteria during vaginal infections and by cadaverine released during the degradation of tissue. Vaginal discharge is caused by the inflammatory reaction in response to infection or foreign bodies in the vagina (contraceptive devices, etc.) or the by-products of the bacteria causing the infection.

Products available in the market relate to feminine hygiene only and do not address the symptomatic treatment of vaginitis. Therefore, there is a need for a product for providing relief from symptoms of vaginitis. The symptoms of vaginitis can be devastating for women to the point that they are not able to perform routine daily activities and are a common reason for visiting the gynecologist.

Vaginitis is an inflammation of the vagina. Common symptoms of vaginitis include discharge, odor and discomfort. Vaginitis often is caused by infections. The symptoms of vaginitis may be incapacitating and are a common reason for visiting the gynecologist and can lead to frustration, embarrassment, anger, lost days from work, marital conflict and loss of ability to enjoy a normal personal, professional and social life.

The most common vaginal infections are bacterial vaginosis, trichomoniasis and vaginal yeast infection or candidiasis. Some vaginal infections are transmitted through sexual contact, but others, such as yeast infections, probably are not.

Although most vaginal infections in women are due to bacterial vaginosis, trichomoniasis or yeast, there may be other causes as well. These causes may include allergic and irritative factors or sexually transmitted diseases such as chlamydia or gonorrhea.

### Bacterial Vaginosis

Bacterial vaginosis (BV) is the most common cause of vaginitis symptoms among women of childbearing age. Previously called nonspecific vaginitis or Gardnerella-associated vaginalis, BV is associated with sexual activity. BV reflects a change in the vaginal ecosystem. This imbalance, including pH changes, occurs when different types of bacteria outnumber the normal ones. Instead of Lactobacillus bacteria being the most numerous of the bacteria, increased numbers of organisms, such as Gardnerella vaginitis, Bacteroides, Mobiluncus and Mycoplasma hominis, are found in the vaginas of women with BV. A change in sexual partners and douching may increase the risk of acquiring bacterial vaginosis. Bacterial vaginosis is associated with Pelvic Inflammatory Disease and complications of pregnancy such as premature labor.

The primary symptom of BV is an abnormal, odorous vaginal discharge. The fish-like odor is noticeable especially after intercouse. A physician may observe these signs during a physical examination and may confirm the diagnosis by testing vaginal fluid.

The test includes examining the sample of vaginal fluid under a microscope to detect the presence of the organisms associated with BV. Diagnosis is based on the absence of lactobacilli, the presence of numerous “clue cells” (cells from the vaginal lining that are coated with BV organisms), a fishy odor and decreased acidity or change in pH of vaginal fluid. Treatment is antibiotics, such as metronidazole or clindamycin.

### Trichomoniasis

Trichomoniasis is a common sexually transmitted disease that affects 2 to 3 million Americans yearly. It is caused by a single-celled protozoan parasite called trichomonas vaginalis.

In women, symptoms usually appear within four to twenty days of exposure. These symptoms include a heavy, yellow-green or gray vaginal discharge, odor and discomfort during intercourse. Diagnosis includes examination of discharge under the microscope and visualization of trichomonas vaginalis or laboratory tests such as cultures. Trichomoniasis can be associated with other sexually transmitted diseases or the complications of pregnancy. Trichomoniasis is treated by metronidazole.

### Vaginal Yeast Infection

Vaginal yeast infection or vulvovaginal candidiasis is a common cause of vaginal irritation. Approximately 75 percent of all women will experience at least one symptomatic yeast infection during their lifetimes. Yeast are always present in the vagina in small numbers, and symptoms only appear with overgrowth. Several factors are associated with increased symptomatic infection in women, including pregnancy, uncontrolled diabetes mellitus and the use of oral contraceptives or antibiotics. Other factors that may increase the incidence of yeast infection include using douches, perfumed feminine hygiene sprays and topical antimicrobial agents and wearing tight, poorly ventilated clothing and underwear. Whether or not yeast can be transmitted sexually is unknown.

The symptoms of yeast infection in women may include discharge, odor, itching and discomfort. The thick, whitish-gray discharge is typically described as cottage-cheese-like in nature, although it can vary from watery to thick in consistency. Most male partners of women with yeast infection do not experience any symptoms of the infection. A transient self-limiting rash and burning sensation of the penis, however, have been reported after intercourse if condoms were not used.

Diagnosis is based upon microscopic examination of vaginal secretions for evidence of yeast forms. Various antifungal vaginal/oral medications (butoconazole, miconazole, clotrimazole, tioconazole and fluconazole) are available to treat yeast infection.

### Chlamydia

This infection is a common bacterial, sexually transmitted disease, with an estimated 4 to 8 million new cases occurring each year. Chlamydial infection may cause an abnormal genital discharge and burning with urination. In women, untreated chlamydial infection may lead to pelvic inflammatory disease, one of the most common causes of ectopic pregnancy and infertility in women. Cultures are used for the diagnosis of chlamydia and the treatment includes antibiotics like Tetracycline.

### Gonorrhea

The symptoms of gonorrhea are a discharge from the vagina, often with an odor and painful or difficult urination. The most common and serious complications occur in women and, as with chlamydial infection, these complications include pelvic inflammatory disease (PID), ectopic pregnancy and infertility. Cultures are used for diagnosis and antibiotics are used for the treatment.

### DESCRIPTION OF THE INVENTION

The present invention contains a unique combination of ingredients to provide relief from vaginal discharge and odor seen during vaginitis. A conventional towelette can be used to apply the composition and cleanse the genital area. The towelette is producted by placing a conventional towelette material, usually 8×5.25 inch woven towelette cloth, into a 2.167×3.5 inch pouch. Then the pouch is filled in a conventional manner with about 6 millileters of the liquid composition of this invention.

### EXAMPLE

In accordance with the present invention, the following ingredients are mixed in the lab in the quantities described below. The mixture is then tested for color, odor, pH, specific gravity and microbial count. Percentages given of ingredients are approximate.

| % by volume | INGREDIENT (INCI NAME) | FUNCTION |
|---|---|---|
| 93.95 | Water | Solvent |
| 5.00 | Saccharomyces Ferment | Bioconversion of |
| | | Odors |
| 0.50 | Octoxynol-9 | Detergent/Emulsifier |
| 0.20 | Potassium Sorbate | Antifungal/ |
| | | Preservative |
| 0.20 | Cetylpyridinium Chloride | Antiseptic |
| 0.10 | Disodium EDTA | Chelator |
| 0.05 | Lactic Acid | Acidifier |
| 100.00 | | |

Alternate ingredients for each function can be used, and exemplary alternate ingredients and their functions are listed below as follows:

Solvents Including Water (92-95% by Volume):

1. Xylene
2. Ethoxydiglycol
3. Alcohol
4. Propylene Gycol
   Odor Control (4-6% by Volume):
1. Potassium Alum
2. Aluminum Citrate
3. Aluminum Bromohydrate
4. Saccharomyces Ferment
5. Dichlorophene
   Emulsifiers (0.4-0.6% by Volume):
1. Lecithin
2. Fatty Alcohols
3. Alkoxylated Carboxylic Acids
4. Alkoxylated Amides
5. Alcoxylated Alcohols
6. Octoxynol-9
   Preservatives (0.15-0.25% by Volume):
1. Alpha Hydroxy Acid
2. Parabens (Methyl Paraben, Ethyl Paraben)
3. Imidazolidinyl Urea
4. Propyl Benzoate
5. Potassium Sorbate
   Antiseptics (0.15-0.25% by Volume):
1. Essential Oils
2. Alpha-Bisabolol
3. Aluminum Diacetate
4. Chlorothymol
5. Cetylpyridinium Chloride
   Chelators (0.04-0.06% by Voume):
1. Trisodium Phosphate
2. Sodium Oxalate
3. Pentetic Acid
4. Bismuth Citrate
5. Disodium EDTA
   Acidifiers (0.04-0.06% by Volume):
1. Citric Acid
2. Acetic Acid
3. Ascorbic Acid
4. Glycolic Acid
5. Lactic Acid

The percentages by volume of each ingredient can be within a range of plus or minus 20% from the exemplary percentages listed above.

The wipe is removed from the packet and unfolded. Feminine area is gently wiped and the wipe is discarded. This can be repeated as needed.

The unique combination of antifungal, antiseptic, detergent and odor bioconverting agents, such as especially saccaromyces ferment, has a synergistic effect resulting in symptomatic relief of vaginitis in women.

1. A device for the treatment of vaginitis comprising an absorbent substrate impregnated with a liquid composition comprising a predominant amount of solvent, 4-6 percent by volume of an odor controlling agent, 0.4-0.6 percent by volume of an emulsifier, 0.15-0.25 percent by volume of a preservative, 0.15-0.25 percent by volume of an antiseptic, 0.04-0.06 percent by volume of a chelating agent, and 0.04-0.06 percent by volume of an acidifer.

2. The device of claim 1 wherein said solvent is selected from one or more of water, xylene, ethoxydiglycol, alcohol and propylene gycol; said odor controlling agent is selected from one or more of potassium alum, aluminum citrate, aluminum bromohydrate, saccharomyces ferment and dichlorophene; said emulsifer is selected from one or more of lecithin, fatty alcohols, alkoxylated carboxylic acids, alkoxylated amides, alcoxylated alcohols and octoxynol-9; said preservatives are selected from one or more of alpha hydroxy acids, alkyl parabens, imidazolidinyl urea, propyl benzoate and potassium sorbate; said antiseptic is selected from one or more of essential oils, alpha-bisabolol, aluminum diacetate, chlorothymol and cetylpyridinium chloride; said chelating agent is selected from one or more of trisodium phosphate, sodium oxalate, pentetic acid, bismuth citrate and disodium ethylene diamine tetraacetic acid; and said acidifiers are selected from one or more of citric acid, acetic acid, ascorbic acid, glycolic acid and lactic acid.

3. The device of claim 1 wherein said absorbent substrate is a fibrous wipe.

4. The device of claim 1 wherein said liquid composition consists essentially of an aqueous solution containing about 4-6 percent by volume saccharomyces ferment, 0.4-0.6 percent by volume oxytoxynol-9, 0.15-0.25 percent by volume potassium sorbate, 0.15-0.25 percent by volume cetylpyridinium chloride, 0.04-0.06 percent by volume disodium EDTA; and 0.04-0.06 percent by volume lactic acid.

5. A method for the treatment of vaginitis which comprises applying to the effected area of the body a liquid composition comprising a predominant amount of solvent, 4-6 percent by volume of an odor controlling agent, 0.4-0.6 percent by volume of an emulsifier, 0.15-0.25 percent by volume of a preservative, 0.15-0.25 percent by volume of an antiseptice, 0.04-0.06 percent by volume of a chelating agent and 0.04-0.06 percent by volume of an acidifier.

6. The method of claim 5 wherein said solvent is selected from one or more of water, xylene, ethoxydiglycol, alcohol and propylene gycol; said odor controlling agent is selected from one or more of potassium alum, aluminum citrate, aluminum bromohydrate, saccharomyces ferment and dichlorophene; said emulsifer is selected from one or more of lecithin, fatty alcohols, alkoxylated carboxylic acids, alkoxylated amides, alcoxylated alcohols and octoxynol-9; said preservatives are selected from one or more of alpha hydroxy acids, alkyl parabens, imidazolidinyl urea, propyl benzoate and potassium sorbate; said antiseptic is selected from one or more of essential oils, alpha-bisabolol, aluminum diacetate, chlorothymol and cetylpyridinium chloride; said chelating agent is selected from one or more of trisodium phosphate, sodium oxalate, pentetic acid, bismuth citrate and disodium ethylene diamine tetraacetic acid; and said acidifiers are selected from one or more of citric acid, acetic acid, ascorbic acid, glycolic acid and lactic acid.

7. The method of claim 5 wherein said liquid composition consists essentially of an aqueous solution containing about 4-6 percent by volume saccharomyces ferment, 0.4-0.6 percent by volume oxytoxynol-9, 0.15-0.25 percent by volume potassium sorbate, 0.15-0.25 percent by volume cetylpyridinium chloride, 0.04-0.06 percent by volume disodium EDTA; and 0.04-0.06 percent by volume lactic acid.

8. The method of claim 5 wherein said liquid composition is impregnated on an absorbent substrate.

9. The method of claim 8 wherein said absorbent substrate is a fibrous wipe.

10. The method of claim 5 wherein said effected area to which the liquid composition is applied is the human female genitalia.
